(19) United States
(12) Patent Application Publication
(10) Pub. No.: US 2020/0360154 A1
(43) Pub. Date: Nov. 19, 2020

(54) INTERVERTEBRAL SPACING INSERT AND INSERTION TOOL THEREOF

(71) Applicant: S Kohan Pty Ltd, Kogarah (AU)

(72) Inventors: Eland CRAMLET, Abbotsford (AU); Saeed KOHAN, Kogarah (AU)

(73) Assignee: S Kohan Pty Ltd, Kogarah (AU)

(21) Appl. No.: 15/931,870

(22) Filed: May 14, 2020

(30) Foreign Application Priority Data
May 14, 2019 (AU) 2019901636

(51) Int. Cl. A61F 2/46; A61F 2/44

(52) U.S. Cl. CPC A61F 2/4611; A61F 2002/4615; A61F 2/442; A61F 2/447

(57) ABSTRACT

An intervertebral spacing insert insertion tool for separating adjacent vertebrae and locating an intervertebral spacing insert within the adjacent vertebrae, the insertion tool includes an adjustable spacing arrangement configured for insertion between the adjacent vertebrae and for spacing the adjacent vertebrae apart to present an intervertebral space and a guide configured for guiding the intervertebral spacing insert into the intervertebral space.

### FIELD

The present invention relates to an intervertebral spacing arrangement and a means for inserting the spacing arrangement between adjacent vertebrae in a patient.

The invention has been developed primarily for use with an intervertebral spacing arrangement in the form of an intervertebral fusion cage and will be described hereinafter with reference to this application. However, it will be appreciated that the invention is not limited to this particular field of use.

### BACKGROUND

Presently, intervertebral spacing arrangements, which may take the form of intervertebral fusion cages are inserted between a patient's adjacent vertebrae typically using force in the form of a surgical mallet or similar device. The intervertebral cage is typically inserted between adjacent vertebrae by striking the intervertebral cage into position between the adjacent vertebrae.

It will be appreciated that such a means of insertion can have adverse consequences, especially if the patient has osteopenia or is suffering from more serious conditions such as osteoporosis in which the patient's bone structure will be brittle and prone to damage due to applied forces which are common with present insertion techniques.

The present invention seeks to provide an alternative to current insertion methods, which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY

According to a first aspect, the present invention may be said to consist in an intervertebral spacing insert insertion tool for separating adjacent vertebrae and locating an intervertebral spacing insert within the adjacent vertebrae, the insertion tool comprising:
a) a spacing arrangement configured for insertion between the adjacent vertebrae and for spacing the adjacent vertebrae apart to present an intervertebral space; and
b) a guide configured for guiding the intervertebral spacing insert into the intervertebral space.

In one embodiment, the insertion tool includes an adjustment mechanism for facilitating adjustment of the spacing between the adjacent vertebrae in use.

In one embodiment, the adjustment mechanism is connected to the spacing arrangement and configured to adjust movement of the spacing arrangement.

In one embodiment, the adjustment mechanism includes a threaded adjustment shaft and at least one complementary threaded aperture.

In one embodiment, the spacing arrangement is adjustable by a ratchet mechanism.

In one embodiment, the spacing arrangement comprises at least two arms movable relative to each other.

In one embodiment, the guide includes a guide formation associated with each of the at least two arms.

In one embodiment, the guiding formations are configured for engagement with complementary guiding formations on the spacing insert.

In one embodiment, the guiding formations extend along at least part of the length of the at least two arms.

In one embodiment, the guiding formations are configured for slidable engagement with complementary guiding formations on the spacing insert.

In one embodiment, the guiding formations are ridges configured for slidable engagement with complementary slot formations on the spacing insert.

In one embodiment, the two arms are pivotably coupled to each other.

In one embodiment, the at least two arms are configured for movement between a first position in which the arms are located proximate each other, and a second position in which the arms are spaced further apart from each other than the first position.

In one embodiment, the spacing arrangement includes a torque transfer arrangement adapted to aid in the rotation of the tool.

In one embodiment, the torque transfer arrangement includes a handle for use by an operator.

In one embodiment, the handle extends substantially transversely from the arms.

In one embodiment, the intervertebral spacing insert is an intervertebral fusion cage.

According to a further aspect, the present invention may be said to consist an intervertebral spacing insert rotating tool for rotating the intervertebral spacing insert in situ when the spacing insert is located between adjacent vertebrae, the rotating tool comprising:
a) engaging formations configured for releasably engaging the spacing insert in situ when the spacing insert is located between the adjacent vertebrae; and
b) a torque transfer arrangement configured for facilitating the rotation of the spacing insert in situ.

In one embodiment, the insertion tool includes a spacing arrangement configured for insertion between the adjacent vertebrae and for spacing the adjacent vertebrae apart to present an intervertebral space.

In one embodiment, the spacing arrangement includes at least two arms movable relative to each other.

In one embodiment, the engaging formations are located at or towards a distal end of the at least two arms.

In one embodiment, the spacing arrangement includes an adjustment mechanism configured for adjustment of the spacing between the at least two arms.

In one embodiment, the at least two arms are pivotably coupled by a pivoting arrangement.

In one embodiment, the torque transfer arrangement includes a handle extending substantially transversely from the at least two arms.

In one embodiment, the adjustment mechanism includes a threaded adjustment shaft.

In one embodiment, the threaded adjustment shaft is lockable, to provide positive engagement of the torque transfer arrangement with the intervertebral spacing insert when in use.

In one embodiment, the engaging formations are adjustable by a ratchet mechanism.

In one embodiment, the at least two arms are configured for movement between a first position in which the arms are located proximate each other, and a second position in which the arms are spaced further apart from each other than the first position.

In one embodiment, the engaging formations act as a guide to guide the intervertebral spacing insert into the intervertebral space between the adjacent vertebrae.

In one embodiment, the guide includes a guide formation associated with each of the at least two arms.

In one embodiment, the guide formation is configured for engagement with complementary guiding formations on the spacing insert.

In one embodiment, the guiding formations extend along at least part of the length of the at least two arms.

In one embodiment, the guiding formations are configured for slidable engagement with complementary guided formations on the spacing insert.

In one embodiment, the guiding formations are ridges configured for slidable engagement with complementary slot formations on the spacing insert.

In one embodiment, the arms and handle are configured as an L-shape.

In one embodiment, the engaging formations are configured to provide a positive engagement of the torque transfer arrangement with the spacing insert during rotation of the spacing insert in use.

In one embodiment, the intervertebral spacing insert is an intervertebral fusion cage.

According to a further aspect, the present invention may be said to consist in an intervertebral spacing insert for use with an insertion tool comprising guiding formations for guiding the spacing insert into an intervertebral space in use, the intervertebral spacing insert comprising:
a) a guided formation configured for movable engagement with the guiding formations on the insertion tool to move along at least part of the length of the insertion tool to locate the intervertebral spacing insert into the intervertebral space in use.

In one embodiment, the guided formation is a slot.

In one embodiment, the intervertebral spacing insert includes a hole for engagement with a control stick.

In one embodiment, the tapered leading end distal or proximal.

In one embodiment, the preceding claims, wherein the intervertebral spacing insert is an intervertebral fusion cage.

According to a further aspect, the present invention may be said to consist in an intervertebral spacing insert for use with an insertion tool for locating the intervertebral spacing insert into an intervertebral space, the intervertebral spacing insert comprising:
a) a torque receiving formation adapted for engaging with the insertion tool and facilitating rotation of the spacing insert by the insertion tool in use.

According to yet a further aspect, the present invention may be said to consist of an intervertebral spacing insert for use with an insertion tool adapted for rotation of the spacing insert upon insertion into an intervertebral space, the spacing insert comprising:
a) an external surface of the spacing insert being adapted for rotation within the intervertebral space with the aid of the insertion tool.

In one embodiment, the insert comprises bevelled edges.

In one embodiment, the intervertebral spacing insert is an intervertebral fusion cage.

According to a further aspect, the present invention may be said to consist in a method for inserting an intervertebral spacing insert using an intervertebral insertion tool between adjacent vertebrae of a patient, the insertion tool comprising a spacing arrangement, the method comprising the steps of:
a) forming an incision to a posterior of an intervertebral space;
b) removing a portion of intervertebral disc from the intervertebral space;
c) inserting the insertion tool between the adjacent vertebrae in the patient;
d) spacing apart the spacing arrangement, to simultaneously separate the adjacent vertebrae and create an intervertebral space;
e) inserting the intervertebral spacing insert into the intervertebral insertion tool;
f) sliding the intervertebral spacing insert along a length of the intervertebral insertion tool and into the intervertebral space;
g) rotating the insertion tool to locate the intervertebral spacing insert into a final location; and
h) disengaging intervertebral insertion tool from intervertebral spacing insert and removing the insertion tool from the patient.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

Other aspects of the invention are also disclosed.

### BRIEF DESCRIPTION OF THE DRAWINGS

Notwithstanding any other forms which may fall within the scope of the present invention, a preferred embodiment of the invention will now be described, by way of example only, with reference to the accompanying drawings in which:

FIG. 1 is a view of the intervertebral spacing insert in accordance with a preferred embodiment of the present invention;

FIG. 2 is a view of an insertion tool in use with the intervertebral spacing insert as shown in FIG. 1 in accordance with another preferred embodiment of the present invention; and

FIG. 3A shows view of the insertion tool and intervertebral spacing insert in use.

FIG. 3B shows view of the insertion tool and intervertebral spacing insert in use.

FIG. 3C shows view of the insertion tool and intervertebral spacing insert in use.

### DETAILED DESCRIPTION

It should be noted in the following description that like or the same reference numerals in different embodiments denote the same or similar features.

A view of an intervertebral spacing insert according to a first aspect of the invention is generally indicated by the numeral 1000.

Furthermore, an associated intervertebral spacing insert insertion tool for use with the intervertebral spacing insert 1000 is generally indicated by the numeral 2000.

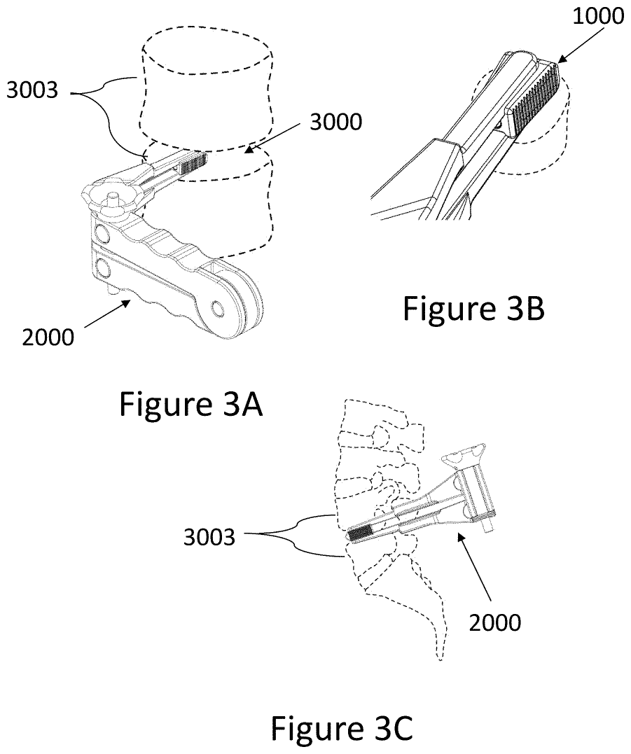

In one embodiment now described, the intervertebral spacing insert 1000 comprises guided formations 101 that are adapted to slidably guide the intervertebral spacing insert 1000 into an intervertebral space 3000 (as shown in FIGS. 3A-3C) when in use with an associated insertion tool 2000 which will be discussed in more detail below.

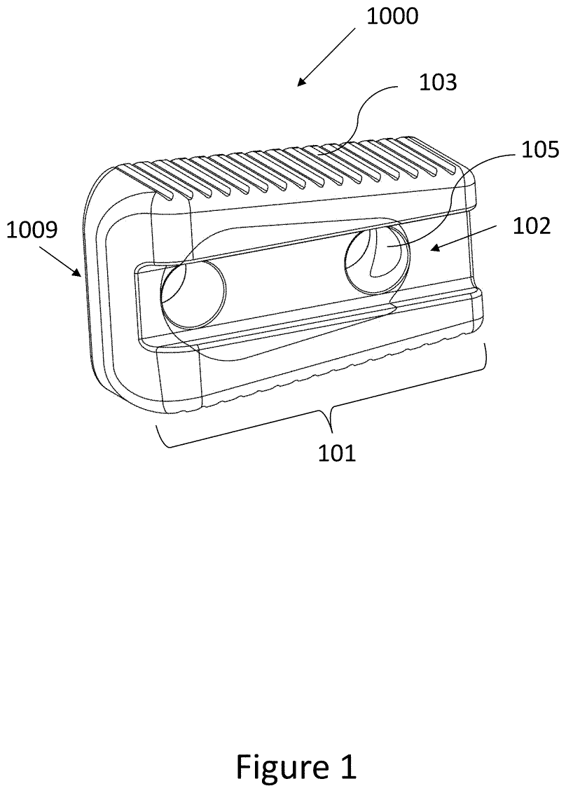

Referring to FIG. 1, the guided formations 101 of the spacing insert 1000 may take a variety of forms. The guided formations 101 as shown in the embodiment in FIG. 1 take the form of slots 102 located on either side of the intervertebral spacing insert 1000. The guided formations 101 extend along the length of the intervertebral spacing insert 1000, providing for slidable movement of the intervertebral spacing insert 1000 when used in combination with the insertion tool 2000.

It will be appreciated by a person skilled in the art, that both the guided formations 101 of the intervertebral spacing insert 1000 and the associated guide 203 of the insertion tool 2000, will desirably will have a smooth profile to aid in the slidable movement of the spacing insert 1000 in respect to the insertion tool 2000 when in use.

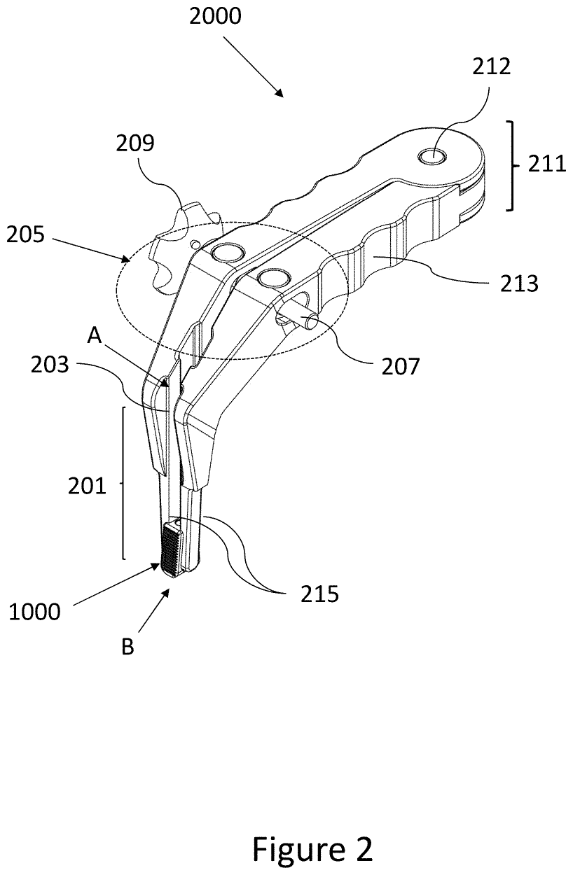

The guided formations 101, which in the embodiment shown take the form of slots 102, are adapted for slidable engagement with the at least one guide 203 of the insertion tool 2000 (shown in FIG. 2). The guided formations 101 have a profile that is complementary in shape to the profile of the guide 203 of the insertion tool 2000. The complementary shape of the guided formations 101 provide for a secure engagement of the spacing insert 1000 with the guide 203 of the insertion tool 2000 while providing for slidable movement of the spacing insert 1000 within the insertion tool 2000.

While the profile of the guide 203 and the guided formations 101 are substantially the same, it is desirable that the mating surfaces of the guided formations 101 and the guide 203 have sufficient spacing between them when in use to allow for slidable movement.

It is envisaged that approximately 0.5 mm-1.5 mm space be provided between the guide 203 and guided formations 101 surfaces to achieve sufficient space to provide for slidable movement of the spacing insert 1000 in relation to the insertion tool 2000. Furthermore, the space provided between the guide 203 and the guided formations 101 should be small enough to ensure accurate guidance and reduced rotational movement of the spacing insert 1000 in relation to the insertion tool 2000 when in use.

It will be appreciated by a person skilled in the art that the guided formations 101 may also take the form of a protrusion (as opposed to the slots 102 shown in the present embodiment) that may be adapted for insertion into a corresponding slot on the insertion tool 2000 with similar effect. In this case, the guided and guiding formations may take a variety of different profiles which allow for slidable movement of the spacing insert 1000 in relation to the associated insertion tool 2000.

However, certain advantages are provided in the use of a slot (shown as 102 in FIG. 1) for the guided formations 101 of the spacing insert 1000, rather than a protrusion. The advantages of using a slot on the spacing insert 1000 include but are not limited to inter alia, reduced size of the spacing insert 1000 and a reduction in the amount of material used in the manufacture of the spacing insert 1000 among other things.

As can be seen in the embodiment shown in FIG. 1, the spacing insert 1000 takes the general form of a intervertebral cage comprising a plurality of projections 103, which may take the form of corrugations as shown in the embodiment in FIG. 1. The plurality of projections 103 are adapted for engaging bone, and providing a surface into which bone may grow once the spacing insert 1000 has been located within an intervertebral space 3000 between a patient's adjacent vertebrae 3003 as is exemplified in FIGS. 3A-3C.

As was previously alluded to, the projections 103 of the spacing insert 1000 may take a number of different forms ranging from corrugations as in the embodiment shown to projections adapted to embed into the bone matrix of the vertebral surface in which they have contact.

FIG. 2 exemplifies the insertion tool 2000 which comprises a spacing arrangement 201 which in the embodiment shown comprises two arms 215 with each arm comprising at least one guide 203 for guiding the intervertebral spacing insert 1000 into an intervertebral space (shown in FIG. 3 as 3000).

The insertion tool 2000 is adjustable using an adjustment mechanism 205 which is adapted to adjust the spacing of the arms 215 of the spacing arrangement 201 to allow for the insertion tool 2000 to be inserted between the adjacent vertebrae 3003 of the patient and subsequently adjusted such that the adjacent vertebrae 3003 may be separated in preparation for insertion of the spacing insert 1000 between the patients adjacent vertebrae 3003.

The adjustment mechanism 205 is connected to the spacing arrangement 201 in the embodiment shown thorough a threaded adjustment shaft 207 that facilitates the separation of the spacing arrangement 201 through the rotation of the threaded adjustment shaft 207. In the embodiment shown, the adjustment of the spacing arrangement is controlled by an operator such as a surgeon, through the rotation of a handle 209 which is connected to the adjustment shaft 207.

As can be seen in FIG. 2, the spacing arrangement 201, which in this embodiment takes the form of two adjustable arms 215 are pivotally coupled to each other through a hinge arrangement 211. The hinge arrangement 211 may take the form of a pin 212 that passes through an extension of each of the adjustable arms 215 allowing for movement of the adjustable arms 215 in relation to each other.

The insertion tool 2000 further includes a torque transfer arrangement 213, which in the embodiment shown takes the form of a handle which extends transversely from the arms 215 of the spacing arrangement 201. The torque transfer arrangement 213 facilitates the rotation of the insertion tool 2000 and hence the spacing insert 1000 when in situ between adjacent vertebrae 3003 as will be explained in more detail below.

It is envisaged that the insertion tool 2000 is constructed from a suitable medical grade material such as titanium or stainless steel with the intention of the insertion tool 2000 to be reused after undergoing sterilization.

As can be seen in FIGS. 3A-3C, the insertion tool 2000 is shown with the spacing insert 1000 positioned to the full extent along the length of the spacing arrangement 201 into its near final position. With reference to FIG. 2, the spacing insert is initially inserted at location A of the insertion tool 2000, by an operator and then slid into place to location B once the insertion tool 2000 has been positioned within the intervertebral space 3000. Once the spacing insert 1000 has been located within the intervertebral space 3000, the operator will then rotate the insertion tool 2000 substantially 90 degrees from its original orientation, locating the spacing insert 1000 such that the plurality of projections 103 of the spacing insert 1000 now face the adjacent vertebrae 3003 of the patient.

The spacing insert 1000 is inserted in the aforementioned manner in order to aid in the insertion of the spacing insert 1000 between the adjacent vertebrae 3003 of the patient with minimal impact and hence damage to the adjacent vertebrae 3003. This is facilitated by the fact that the height of the spacing insert 1000 is at its minimum at insertion and only after it has been inserted between the patients vertebrae 3003 is it rotated into position, ensuring that the plurality of projections 103 face the bone into which it is to embed.

Inserting the spacing insert 1000 in this manner further avoids the abrasive effects of inserting the spacing insert 1000 while in its final orientation, reducing resistance to insertion within the intervertebral space 3000.

It will be appreciated by those skilled in the art, that the spacing insert 1000 is required to be constructed from a biocompatible material due to its location within the patient's body. Such materials may include but are not limited to titanium and composites thereof, polyether ether ketone (PEEK) and other biocompatible materials that exhibit the required strength given its intended purpose.

There are a number of features that distinguish the spacing insert 1000 associated with the present invention from a conventional spacing insert. As was previously mentioned, the spacing insert 1000 comprises guided formations 101, which in the embodiment shown, are two adjacent tracks in the form of slots 102. The slots 102 have been adapted for use with an associated insertion tool 2000.

To aid in the insertion of the spacing insert 1000 with minimal impact on adjacent bone, the spacing insert 1000 further comprises a tapered leading end 1009 which has a smooth tapered profile further aiding in the insertion between the adjacent vertebrae 3003. In order to assist with the accurate positioning of the spacing insert 1000 into adjacent vertebrae 3003 the spacing insert 1000 further comprises a threaded hole 105 for use with a control stick (not shown), which is used to guide the spacing insert 1000 into its final location between the adjacent vertebrae 3003.

In use, the patient's spine is exposed through the creation of an access hole, through the use of a scalpel, or lancet permitting access to the patient's spine. The vertebrae 3003 in which the spacing insert 1000 is to be inserted are located and the surgeon then proceeds to remove at least a portion of the patient's intervertebral disc material from between the adjacent vertebrae 3003, providing room for the intervertebral spacing insert 1000 to be inserted.

The insertion tool 2000 may then be inserted between adjacent vertebrae 3003 in the patient and adjusted to increase the spacing between the adjacent vertebrae 3003 to form an enlarged intervertebral space 3000 and thereby allowing for insertion of the intervertebral spacing insert 1000 into position between adjacent vertebrae 3003.

The insertion of the intervertebral spacing insert 1000 is achieved by inserting the intervertebral spacing insert 1000 into position A of the insertion tool 2000 (as shown in FIG. 2), and using a sliding action to slide the spacing insert 1000 into position B of the insertion tool 2000 (as show in FIG. 2), with the aid of a control stick (not shown) which is used to guide the spacing insert from position A to position B on the insertion tool 2000.

As is exemplified in FIGS. 3A-C, the intervertebral space 3000 that exists between the adjacent vertebrae 3003 is enlarged by opening the spacing arrangement 201, this is achieved by the rotational adjustment of the handle 209 to achieve the desired spacing. Once the intervertebral spacing insert 1000 has been inserted into the intervertebral space 3000 by moving the spacing insert from position A to position B of the insertion tool 2000, the insertion tool 2000 is then rotated by approximately 90 degrees to position the spacing insert 1000 into its final position between the patient's adjacent vertebrae 3003. Once the spacing insert 1000 has been positioned in its final location between the adjacent vertebrae 3003, the insertion tool 2000 is then disengaged from the spacing insert 1000 and removed from the patient's body.

It is envisaged that the method of inserting the intervertebral spacing insert 1000 comprises the steps of firstly, forming an incision into the patient to reveal a posterior of an intervertebral space 3000 wherein affected intervertebral disc is removed from between the adjacent vertebrae 3003 in which the spacing insert 1000 is to be inserted. The insertion tool 2000 is then inserted into the intervertebral space 3000 which has been created through the removal of the affected disc. Once the insertion tool 2000 has been inserted within the intervertebral space 3000, the spacing arrangement 201 is spaced apart by use of the adjustment mechanism 205, and once the spacing is sufficient to allow for insertion of the spacing insert 1000 into the insertion tool 2000, the operator such as a surgeon inserts the intervertebral spacing insert 1000 into the insertion tool 2000 at location A, wherein the operator slides the spacing insert 1000 along the length of the insertion tool 2000 to position B and into position within the patient's intervertebral space 3000. Once the spacing insert 1000 has been inserted within the intervertebral space 3000, the insertion tool 2000 is then rotated approximately 90 degrees to place the intervertebral spacing insert into its final location within the patient's intervertebral space 3000. After which the insertion tool 2000 is removed from the patient and the incision is closed.

### Interpretation

### Markush Groups

In addition, where features or aspects of the invention are described in terms of Markush groups, those skilled in the art will recognise that the invention is also thereby described in terms of any individual member or subgroup of members of the Markush group.

### Chronological Sequence

For the purpose of this specification, where method steps are described in sequence, the sequence does not necessarily mean that the steps are to be carried out in chronological order in that sequence, unless there is no other logical manner of interpreting the sequence.

### Embodiments

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Terminology

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

For the purposes of this specification, the term “plastic” shall be construed to mean a general term for a wide range of synthetic or semisynthetic polymerization products, and generally consisting of a hydrocarbon-based polymer.

As used herein the term “and/or” means “and” or “or”, or both.

As used herein “(s)” following a noun means the plural and/or singular forms of the noun.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the biomedical industries.

The claims defining the invention are as follows:

1. An intervertebral spacing insert insertion tool for separating adjacent vertebrae and locating an intervertebral spacing insert within the adjacent vertebrae, the insertion tool comprising:
a) a spacing arrangement configured for insertion between the adjacent vertebrae and for spacing the adjacent vertebrae apart to present an intervertebral space; and
b) a guide configured for guiding the intervertebral spacing insert into the intervertebral space.

2. The insertion tool as claimed in claim 1, wherein the insertion tool includes an adjustment mechanism for facilitating adjustment of the spacing between the adjacent vertebrae in use.

3. The insertion tool as claimed in claim 2, wherein the adjustment mechanism is connected to the spacing arrangement and configured to adjust movement of the spacing arrangement.

4. The insertion tool as claimed in claim 3, wherein the adjustment mechanism includes a threaded adjustment shaft and at least one complementary threaded aperture.

5. The insertion tool as claimed in claim 3, wherein the spacing arrangement is adjustable by a ratchet mechanism.

6. The insertion tool as claimed in claim 1, wherein the spacing arrangement comprises at least two arms movable relative to each other.

7. The insertion tool as claimed in claim 6, wherein the guide includes a guide formation associated with each of the at least two arms.

8. The insertion tool as claimed in claim 6, wherein the guiding formations are configured for engagement with complementary guiding formations on the spacing insert.

9. The insertion tool as claimed in claim 6, wherein the at least two arms are configured for movement between a first position in which the arms are located proximate each other, and a second position in which the arms are spaced further apart from each other than the first position.

10. The insertion tool as claimed in claim 1, wherein the spacing arrangement includes a torque transfer arrangement adapted to aid in the rotation of the tool.

11. The insertion tool as claimed in claim 10, wherein the torque transfer arrangement includes a handle for use by an operator.

12. The insertion tool as claimed in claim 1, wherein the intervertebral spacing insert is an intervertebral fusion cage.

13. An intervertebral spacing insert rotating tool for rotating the intervertebral spacing insert in situ when the spacing insert is located between adjacent vertebrae, the rotating tool comprising:
a) engaging formations configured for releasably engaging the spacing insert in situ when the spacing insert is located between the adjacent vertebrae; and
b) a torque transfer arrangement configured for facilitating the rotation of the spacing insert in situ.

14. The insertion tool as claimed in claim 13, wherein the insertion tool includes a spacing arrangement configured for insertion between the adjacent vertebrae and for spacing the adjacent vertebrae apart to present an intervertebral space.

15. The insertion tool as claimed in claim 14, wherein the spacing arrangement includes at least two arms movable relative to each other.

16. The insertion tool as claimed in claim 15, wherein the engaging formations are located at or towards a distal end of the at least two arms.

17. The insertion tool as claimed in claim 16, wherein the spacing arrangement includes an adjustment mechanism configured for adjustment of the spacing between the at least two arms.

18. The insertion tool as claimed in claim 17, wherein the at least two arms are pivotably coupled by a pivoting arrangement.

19. The insertion tool as claimed in claim 18, wherein the torque transfer arrangement includes a handle extending substantially transversely from the at least two arms.

20. The insertion tool as claimed in claim 17, wherein the adjustment mechanism includes a threaded adjustment shaft.

21. The insertion tool as claimed in claim 20, wherein the threaded adjustment shaft is lockable, to provide positive engagement of the torque transfer arrangement with the intervertebral spacing insert when in use.

22. The insertion tool as claimed in claim 21, wherein the engaging formations are adjustable by a ratchet mechanism.
